(19) United States
(12) Patent Application Publication
(10) Pub. No.: US 2014/0303185 A1
(43) Pub. Date: Oct. 9, 2014

(54) NOVEL POLYMORPHS OF VILAZODONE HYDROCHLORIDE

(71) Applicant: ORCHID CHEMICALS & PHARMACEUTICALS LIMITED, Chennai (IN)

(72) Inventors: Buchi Reddy REGURI, Chennai (IN); Venkateshwar Goud THIRUMANI, Chennai (IN); Srinivasa Rao MANNAVA, Chennai (IN); Suresh DEVINENI, Chennai (IN)

(21) Appl. No.: 14/249,064

(22) Filed: Apr. 9, 2014

(30) Foreign Application Priority Data
Apr. 9, 2013 (IN) 1608/CHE/2013

(51) Int. Cl. C07D 405/12

(52) U.S. Cl. CPC C07D 405/12
USPC 514/254.09; 544/373

(57) ABSTRACT

The present invention relates to novel crystalline polymorphic forms of Vilazodone hydrochloride of formula (I). This present invention further provides the process for the preparation of novel polymorphic forms of Vilazodone hydrochloride.

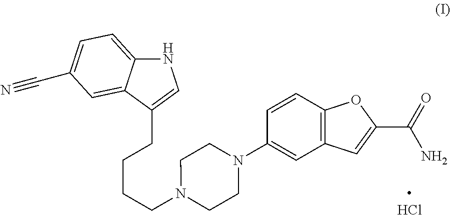 [SMILES: N#Cc1ccc2[nH]cc(CCCCN3CCN(c4ccc5oc(C(N)=O)cc5c4)CC3)c2c1]

### FIELD OF THE INVENTION

The present invention relates to novel polymorphic forms of Vilazodone hydrochloride of formula (I). This present invention further provides the process for the preparation of novel polymorphic forms of Vilazodone hydrochloride.

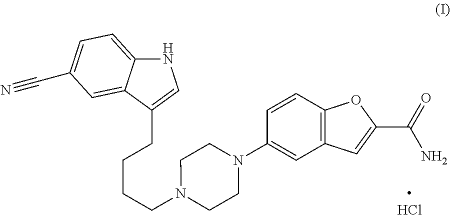 [SMILES: N#Cc1ccc2[nH]cc(CCCCN3CCN(c4ccc5oc(C(N)=O)cc5c4)CC3)c2c1]

### BACKGROUND OF THE INVENTION

Vilazodone is a serotonergic antidepressant which is marketed in United States of America under the brand name Viibryd. Compound of formula (I) are active on the central nervous system, especially in terms of 5-HT₁A-agonist and 5-HT-reuptake inhibition. The chemical name for Vilazodone is 1-[4-(5-cyanoindol-3-yl)butyl]-4-(2-carbamoylbenzofuran-5-yl) piperazine hydrochloride.

U.S. Pat. No. 5,532,241 describes the preparation of 1-[4-(5-cyanoindol-3-yl)butyl]-4-(2-carbamoyl-benzofuran-5-yl)-piperazine hydrochloride by reacting 1-[4-(5-cyanoindol-3-yl)butyl]-4-(2-carboxybenzofuran-5-yl)piperazine with 2-chloro-1-methylpyridinium methanesulfonate (CMPM) in N-methylpyrrolidine followed by NH₃ as depicted scheme 1; Customary working up gives the free base 1-[4-(5-cyanoindol-3-yl)butyl]-4-(2-carboxybenzofuran-5-yl)piperazine.

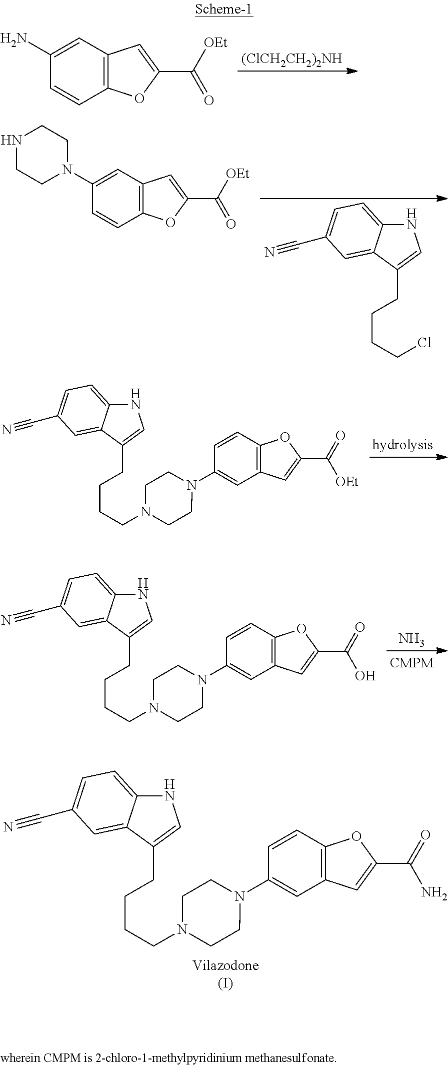

U.S. Pat. No. 7,381,726 and its continuation patents/publications provides the crystalline modifications of 1-[4-(5-cyanoindol-3-yl)butyl]-4-(2-carbamoyl-benzofuran-5-yl)-piperazine dihydrochloride, six (five+dihydrochloride XIII) new forms of 1-[4-(5-Cyanoindol-3-yl)butyl]-4-(2-carbamoyl-benzofuran-5-yl)-piperazine hydrochloride, three new forms of 1-[4-(5-cyanoindol-3-yl)butyl]4-(2-carbamoyl-benzofuran-5-yl)-piperazine hydrochloride hydrate, six new forms of solvates of 1-[4-(5-cyanoindol-3-yl)butyl]4-(2-carbamoyl-benzofuran-5-yl)-piperazine hydrochloride and pure amorphous 1-[4-(5-cyanoindol-3-yl)butyl]-4-(2-carbamoyl-benzofuran-5-yl)-piperazine hydrochloride have been disclosed. These forms are referred to as I, II, III, IV, V, VI, VII, VIII, IX, X, XI, XIII, XIV, XV and XVI respectively. This patent also describes the preparation of said crystalline modifications. This patent disclose that following the procedure reported in U.S. '241 results with mixture of free base of vilazodone and its HCl. According to this patent said mixture is prepared by treating the base in 30 ml 2-propanol with 0.1 n 2-propanolic HCL-solution (Merck-Art. No. 1.00326) until precipitation of hydrochloride is complete. The 1-[4-(5-cyanoindol-3-yl)butyl]-4-(2-carbamoyl-benzofuran-5-yl)-piperazine hydrochloride obtained by this process having a melting point of 269-272° C.

WO 2013/078361 discloses new solid state form of vilazodone freebase and also discloses several solid state crystalline forms of vilazodone hydrochloride. The disclosed Vilazodone hydrochloride crystalline forms are named such as form alpha, form beta, form gamma, form delta, form epsilon, form eta, form theta, form lota, form kappa, form lambda, form Mu, form Nu, form zeta, form Xi, form omicron, form pi, form Rho, form sigma, forma tau and also amorphous form of vilazodone hydrochloride.

1632/CHE/2013 describes a process for preparation of vilazodone hydrochloride crystalline form D in non-aqueous medium by treating the solution of the vilazodone freebase in isopropanol with trimethylsilyl chloride (TMSCl).

WO 2013/164794 (IN 1382/DEL/2012) describes three different crystalline forms of vilazodone hydrochloride namely form A, form B & form C and also provides a processes for its preparation.

WO 2013/168126 (IN 1885/CHE/2012) describes several crystalline forms of vilazodone hydrochloride and Vilazodone freebase. The crystalline forms of vilazodone hydrochloride which disclosed in this application named as form B, form C, form D, form E, form F, form G, form H and form I. It also provides process for the preparation of said crystalline forms.

WO 2013/182946 (IN 1743/DEL/2012) discloses three crystalline forms of vilazodone freebase and four crystalline forms of vilazodone hydrochloride.

WO2012131706 (IN 167/MUM/2011) discloses amorphous vilazodone hydrochloride and process for its preparation.

WO2013/088373 (IN 3608/DEL/2011) describes the stable amorphous form of vilazodone hydrochloride and its preparation by utilizing the solvent mixture like methanol-water, ethanol-water and 2-propanol-water and then obtained product were isolated by spray drying or buchi rotavapor.

U.S. Publication No. 2013/0324554 (IN 2242/CHE/2012) describes the amorphous form of vilazodone hydrochloride and its preparation and also describes amorphous co-precipitate of vilazodone hydrochloride with polyvinyl pyrrolidone or with Hypromellose Pthalate or with Hydroxypropyl Cellulose.

However considering the pharmaceutical importance of Vilazodone hydrochloride, still there is a need to develop a different polymorphic forms of Vilazodone hydrochloride which possesses the enhanced stability and purity.

In general, formation of polymorph is influenced by solvent selection, temperature of the solution, rate of stirring, rate of precipitation, mode of mixing, rate of addition of the mixing of solvents and time of stirring. The prediction and formation of polymorphism is affected even by varying the reaction condition, for example different polymorphs can be isolated from the same solvent system by simply stirring the mixture for different period of times, mixing different solvents etc. In the present case, applicant found that use of solvent system DMF with alcohol results in the different pattern (novel polymorph) over the prior art form which is obtained using the solvent DMF. The said novel form obtained by the present invention found to be possessing greater stability over the prior art form. Hence the prediction of stable form with pharmaceutical advantage requires intense research and inventive step.

With our continued intense and diligent work on polymorphic forms of Vilazodone hydrochloride of formula (I), we have discovered new polymorphic forms of Vilazodone HCl herein designated as form A and form B which can be isolated from ecological friendly solvents.

### OBJECTIVES

The primary objective of the present invention is to provide novel polymorphic forms of Vilazodone hydrochloride of formula (I) having good storage stability.

Another objective of the present invention is to provide a robust process for the preparation of novel polymorphic forms of Vilazodone hydrochloride of formula (I).

Yet another objective of the present invention is to provide a pharmaceutical composition containing polymorph of Form A or Form B of Vilazodone HCl.

### SUMMARY

Accordingly the primary aspect of the present invention provides the novel polymorphic form A having substantially the same X-ray diffractogram as set out in FIG. 1. and crystalline form B of Vilazodone hydrochloride having substantially the same X-ray diffractogram as set out in FIG. 2.

Another aspect of the present invention provides a process for the preparation of novel polymorphic form A of Vilazodone hydrochloride, which comprising the steps of:
i) obtaining the solution of Vilazodone hydrochloride in a mixture of halogenated hydrocarbon and alcohol;
ii) adding HCl to reaction mixture of step (i) or vice-versa; and
iii) isolating vilazodone hydrochloride of form A.

Yet another aspect of the present invention provides a process for the preparation of novel polymorphic form B of Vilazodone hydrochloride, which comprising the steps of:
a) obtaining the solution of Vilazodone hydrochloride in polar aprotic solvent;
b) adding the step (a) solution to a an alcoholic HCl solution or vice versa; and
c) isolating vilazodone hydrochloride of form B.

### DRAWINGS

FIG. 1: Illustrates the X-ray powder diffraction pattern of Form A of vilazodone hydrochloride of the present invention.

FIG. 2: Illustrates the X-ray powder diffraction pattern of Form B of vilazodone hydrochloride of the present invention.

The PXRD of above compounds were analyzed using following condition:

Make: BRUKER AXS

Model: D8 ADVANCE

Data handling system: EVA 12.0.0.0.

Anode: COPPER

Radiation: COPPER K alpha-1

Wavelength: 1.5406°

Current & voltage: 40 kV 30 mA

### DETAILED DESCRIPTION

In primary embodiment of the present invention, the solution of vilazodone in solvent(s) in step (i) and step (a) can be obtained by 1) dissolution of vilazodone in solvent or mixture of solvents, 2) extraction of vilazodone from reaction mixture using solvent, optionally concentrated and 3) Vilazodone directly obtained from reaction mixture, optionally concentrated.

In second embodiment of the present invention, the halogenated hydrocarbon solvents used in step (i) is selected from group consisting of dichloromethane, dichloroethane, chloroform and the like; alcohol solvent used in step (i) is selected from group consisting of methanol, ethanol, propanol, isopropanol, isobutanol and the like; preferably a mixture of dichloromethane and methanol.

In third embodiment of the present invention, the HCl used in step (ii) can be added as in the form of gas or in the form of liquid such as concentrated HCl (35% HCL in water), alcoholic HCl, HCl in ethyl acetate, HCl in ether and the like.

In fourth embodiment of the present invention, the alcoholic HCl used in step (b) or step (ii) as stated above is selected from group consisting of methanolic HCl, ethanolic HCl, propanolic HCl, isopropanolic HCl and the like.

In fifth embodiment of the present invention, the polar aprotic solvent used in step (a) is selected from group consisting of dimethylformamide (DMF), dimethylacetamide (DMAc), dimethylsulfoxide (DMSO), sulfolane and the like.

In sixth embodiment of the present invention, the isolation of precipitated or crystallized product of step (iii) and step (c) can be done by conventional techniques such as filteration, decantation and centrifugation.

Seventh embodiment of the present invention describes the novel crystalline polymorphic form A and form B. These forms differ from each other in their physical properties, spectral data and methods of preparation and characterized by their X-ray powder diffraction patterns, Thermo gravimetric analysis (TGA) and/or by their infra red absorption spectrum (IR). The process discloses in the present invention for the preparation of novel form is simple, cost effective, high yielding, precise, reproducible, environment friendly and easy to scale up for industrial manufacture while maintaining the quality of the title product. The processes for the novel forms of vilazodone hydrochloride of present invention have merits of residual solvent limits, which are well below ICH guidelines.

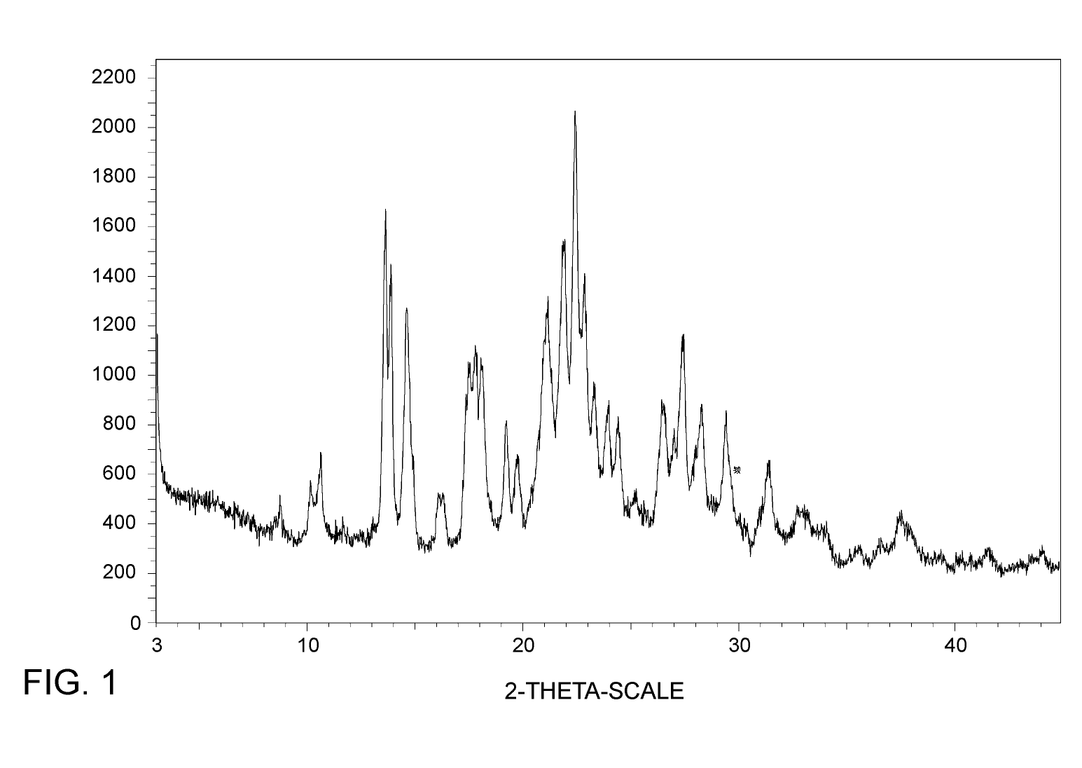

Polymorph form A of Vilazodone hydrochloride is characterized by powder X-ray diffraction pattern as shown in FIG. 1 with major peaks shown in table 1 which list the 2θ and relative intensities.

TABLE 1

| S. No. | 2θ values | Relative intensities (%) |
|---|---|---|
| 1. | 6.648 | 23 |
| 2. | 8.695 | 23.7 |
| 3. | 10.125 | 27.6 |
| 4. | 10.576 | 32.6 |
| 5. | 13.581 | 79.7 |
| 6. | 13.829 | 68.4 |
| 7. | 14.59 | 61.7 |
| 8. | 16.251 | 25.1 |
| 9. | 17.503 | 50.4 |
| 10. | 17.771 | 53.7 |
| 11. | 18.065 | 50.5 |
| 12. | 19.204 | 39.2 |
| 13. | 19.707 | 32.4 |
| 14. | 21.108 | 62.4 |
| 15. | 21.871 | 73.9 |
| 16. | 22.407 | 100 |
| 17. | 22.827 | 66.8 |
| 18. | 23.274 | 46 |
| 19. | 23.919 | 41.9 |
| 20. | 24.383 | 39.5 |
| 21. | 25.204 | 25.1 |
| 22. | 26.486 | 42.5 |
| 23. | 27.002 | 37.1 |
| 24. | 27.393 | 55.6 |
| 25. | 28.261 | 42.6 |
| 26. | 29.389 | 39.9 |
| 27. | 30.303 | 19.3 |
| 28. | 30.989 | 21.1 |
| 29. | 31.381 | 31.7 |

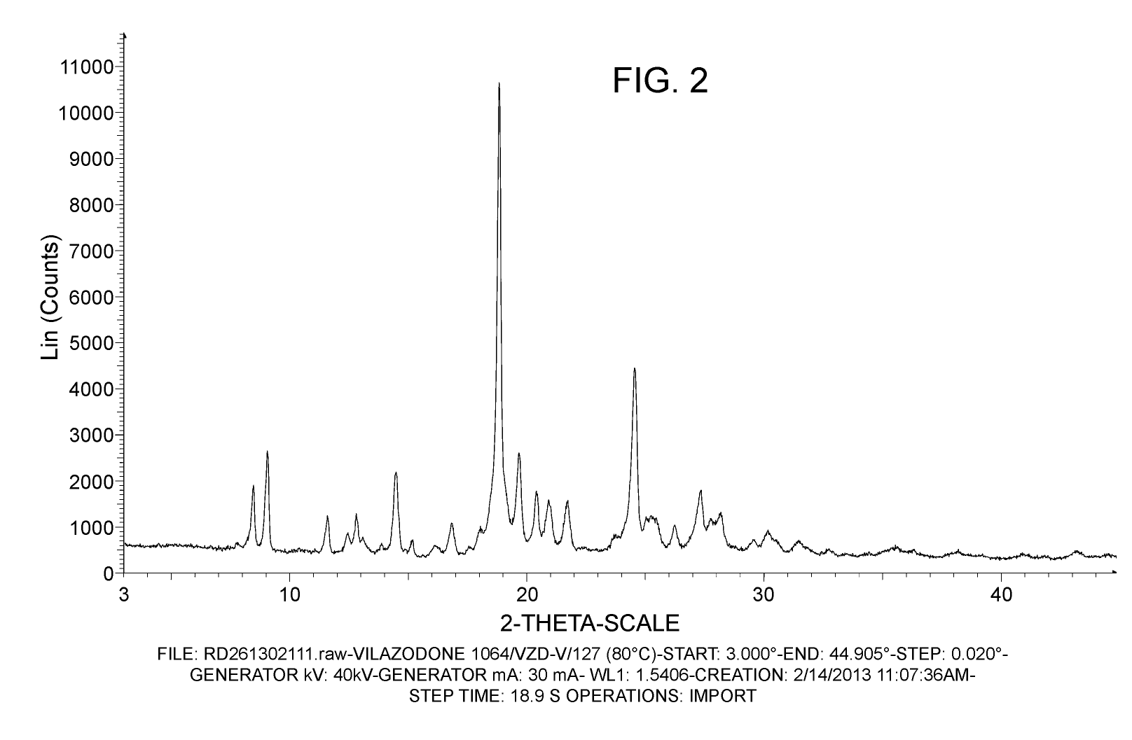

Crystalline polymorph form B of Vilazodone hydrochloride is characterized by powder X-ray diffraction pattern as shown in FIG. 2 with major peaks shown in table 2 which list the 2θ and relative intensities.

TABLE 2

| S. No. | 2θ values | Relative intensities (%) |
|---|---|---|
| 1. | 8.41 | 17.6 |
| 2. | 9.007 | 24.8 |
| 3. | 11.552 | 11.2 |
| 4. | 12.788 | 11.5 |
| 5. | 14.443 | 20.3 |
| 6. | 16.803 | 10 |
| 7. | 18.022 | 8.9 |
| 8. | 18.809 | 100 |
| 9. | 19.642 | 24.4 |
| 10. | 20.39 | 16.3 |
| 11. | 20.914 | 14.7 |
| 12. | 21.681 | 14.5 |
| 13. | 24.54 | 41.9 |
| 14. | 25.042 | 11.1 |
| 15. | 25.243 | 11.5 |
| 16. | 26.225 | 9.5 |
| 17. | 27.31 | 16.8 |
| 18. | 27.753 | 10.9 |
| 19. | 28.155 | 11.9 |
| 20. | 30.163 | 8.4 |

Further Vilazodone hydrochloride obtained as per the present invention can be further micronized, milled or sieved to get the desired particle size required for pharmaceutical composition to achieve the desired dissolution profile. Vilazodone hydrochloride prepared by the present invention is a free flow solid and suitable for pharmaceutical composition.

In another embodiment of the present invention the present invention, the polymorphic form A or form B of vilazodone HCl prepared according to the present invention can be used to prepare pharmaceutical composition comprising vilazodone HCl and pharmaceutical acceptable carrier/excipient and/or diluent.

Starting materials of the present invention can be obtained by the conventional methods reported in the prior arts or by following the methods described in our co-pending application number IN 2032/CHE/2012.

The following examples are provided by way of illustration only and should not be construed to limit the scope of the invention.

### EXAMPLES

### Examples

### Preparation of Vilazodone Hydrochloride (Form A)

### Example-1 a:

To a mixture of dichloromethane (150 ml) and methanol (50 ml), Vilazodone (5 g) was added. The reaction mass was heated to 50-55° C. and stirred till completion of dissolution. To the reaction mass 1N isopropanolic HCl (150 ml) was gradually added at room temperature and stirred. The solid formed was filtered and washed with the mixture of dichloromethane and methanol then dried at 60-65° C. to yield the polymorph form A of Vilazodone hydrochloride.
Yield: 91%
DSC: 287.20° C.
Purity: 99.6% (HPLC Purity)
Moisture content: 2.02%
Residual solvent: Dichloromethane-ND (Not detected), Methanol-ND, Isopropanol-920 ppm.

### Example-1b:

To a mixture of dichloromethane (150 ml) and IPA (50 ml), Vilazodone (5 g) was added. The reaction mass was heated to 50-55° C. and stirred till completion of dissolution. To the reaction mass methanolic HCl was slowly added at room temperature and stirred. The solid formed was filtered and washed with the mixture of dichloromethane and methanol then dried at 60-65° C. to yield the polymorph form A of Vilazodone hydrochloride.
Purity: 99.65% (HPLC Purity)
Moisture Content: 1.10%
Residual solvent: Dichloromethane-33 ppm, Methanol-ND (Not detected), Isopropanol-347 ppm

### Example-1c:

To a mixture of dichloromethane (150 ml), methanol (50 ml) and IPA (120 ml), Vilazodone (5 g) was added. The reaction mass was heated to 50-55° C. and stirred till completion of dissolution. To the reaction mass HCl gas was purged at room temperature and stirred. The solid formed was filtered and washed with the mixture of dichloromethane and methanol then dried at 60-65° C. to yield the polymorph form A of Vilazodone hydrochloride.
Purity: 99.64% (HPLC Purity)

### Examples

### Preparation of Vilazodone Hydrochloride (Form B)

### Example 2a:

To dimethylformamide (15 ml) was added Vilazodone (5 g) and stirred to get clear solution. This reaction mixture was slowly added to methanolic HCl solution (34 ml of 0.25N HCl in methanol) at room temperature. The solid obtained was filtered and washed with methanol, finally dried at 60-65° C. to yield the polymorph form B of Vilazodone hydrochloride.
Yield: 55%
DSC: 288.3° C.
Purity: 99.63% (HPLC Purity)
Moisture content: 2.06%
Residual solvent: Methanol-ND (Not detected), Dimethylformamide-1067 ppm

### Example 2b:

To dimethylformamide was added Vilazodone (5 g) and stirred to get clear solution. To the reaction mixture, methanolic HCl (34 ml of 0.25N HCl in methanol) solution was slowly added under stirring at room temperature. The solid obtained was filtered and washed with methanol, finally dried at 60-65° C. to yield the polymorph form B of Vilazodone hydrochloride.
Yield: 76%
Purity: 99.62% (HPLC Purity)

The following table 3 & 4 provides stability data of Vilazodone HCl prepared according to this invention. From this table it is evident that the novel Form A and Form B prepared according to this invention posses good storage stability at accelerated condition.

TABLE 3
Stability at 40 ± 2° C. & 75 ± 5% RH

| | Form A | | Form B | |
|---|---|---|---|---|
| Description | Initial | Final | Initial | Final |
| Description | white to | white to off | white to off | white to off |
| | off white | white | white | white |
| Purity | 99.65% | 99.60% | 99.62% | 99.60% |
| Moisture content | 1.10% | 1.29% | 2.06% | 1.91% |

TABLE 4
Stability at 60 ± 2° C.

| | Form A | | Form B | |
|---|---|---|---|---|
| Description | Initial | Final | Initial | Final |
| Description | white to | white to off | white to off | white to off |
| | off white | white | white | white |
| Purity | 99.65% | 99.65% | 99.62% | 99.60% |
| Moisture content | 1.10% | 0.76 | 2.06% | 0.81% |

### ADVANTAGES OF THE PRESENT INVENTION

Novel crystalline polymorphic forms of the present invention possess good stability as evidence by its HPLC purity.
Novel crystalline polymorphic forms obtained according to the present invention are free flow in nature, reproducible and makes good ease of handle in commercial scale.
Novel crystalline polymorphic forms obtained according to the present invention has higher solubility when compared to the forms described in the prior art, for example has solubility greater than 0.2 μg/ml, preferably greater than 0.3 μg/ml and hence found to be preferable form for pharmaceutical preparation.

1. A crystalline Vilazodone HCl Form B having X-ray diffraction pattern, which comprises 2θ values (Cu K alpha⁻¹λ=1.54056 Å°) of 8.41, 9.007, 10.365, 11.032, 11.552, 12.788, 14,443, 16.803, 17.561, 18.022, 18.809, 19.642, 20.39, 20.914, 21.681, 24.54, 25.042, 25.243, 26.225, 27.31, 27.753, 28.155and 30.163±0.2.

2. (canceled)

3. A process for the preparation of Vilazodone hydrochloride polymorphic form B having X-ray diffraction pattern, which comprises 2θ values (Cu K alpha⁻¹λ=1.54056 Å°) of 8.41, 9.007, 10.365, 11.032, 11.552, 12.788, 14.443, 16.803, 17.561, 18.022, 18.809, 19.642, 20.39, 20.914, 21.681, 24.54, 25.042, 25.243, 26.225, 27.31, 27.753, 28.155and 30.163±0.2, the process comprising the steps of:
obtaining the solution of Vilazodone hydrochloride in dimethylformamide;
adding alcoholic HCl solution to the reaction mixture of step (a); and
isolating vilazodone hydrochloride of form B.

4. A crystalline Vilazodone HCl Form A having X-ray diffraction pattern, which comprises 2θ values (Cu K alpha⁻¹λ=1.54056 Å°) of 6.648, 8.695, 10.125, 10.576, 13.581, 13.829, 14.59, 16.251, 17.503, 17.771, 18.065, 19.204, 19.707, 21.108, 21.871, 22.407, 22.827, 23.274, 23.919, 24.383, 25.204, 26.486, 27.002, 27.393, 28.261, 29.389, 30.303, 30.989 and 31.381±0.2.

5. (canceled)

6. A process for the preparation of Vilazodone hydrochloride polymorphic form A having X-ray diffraction pattern, which comprises 2θ values (Cu K alpha⁻¹λ=1.54056 Å°) of 6.648, 8.695, 10.125, 10.576, 13.581, 13.829, 14.59, 16.251, 17.503, 17.771, 18.065, 19.204, 19.707, 21.108, 21.871, 22.407, 22.827, 23.274, 23.919, 24.383, 25.204, 26.486, 27.002, 27.393, 28.261, 29.389, 30.303, 30.989 and 31.381±0.2., the process comprising the steps of:
obtaining the solution of Vilazodone hydrochloride in a mixture of halogenated hydrocarbon and alcohol;
adding alcoholic HCl to reaction mixture of step (i); and
isolating Vilazodone hydrochloride of form A.

7. The process as claimed in claim 6, wherein the halogenated hydrocarbon solvents is selected from group consisting of dichloromethane, dichloroethane and chloroform.

8. The process as claimed in claim 6, wherein the alcohol solvent is selected from group consisting of methanol, ethanol, propanol, isopropanol and isobutanol.

9. The process as claimed in claim 3, wherein alcoholic HCl is selected from methanolic HCl, ethanolic HCl, propanolic HCl and isopropanolic HCl.

10. A pharmaceutical composition comprising vilazodone HCl of claim 1 along with pharmaceutical carrier and/or diluent.

11. The process as claimed in claim 6, wherein alcoholic HCl is selected from methanolic HCl, ethanolic HCl, propanolic HCl and isopropanolic HCl.

12. A pharmaceutical composition comprising vilazodone HCl of claim 4 along with pharmaceutical carrier and/or diluent.

13. The process as claimed in claim 6, wherein the halogenated hydrocarbon solvent is dichloromethane.
